(19) United States
(12) Patent Application Publication
(10) Pub. No.: US 2024/0279225 A1
(43) Pub. Date: Aug. 22, 2024

(54) COMPOUNDS AND METHODS FOR THERANOSTIC TARGETING OF PARP ACTIVITY

(71) Applicant: BOARD OF REGENTS, THE UNIVERSITY OF TEXAS SYSTEM, Austin, TX (US)

(72) Inventors: Federica PISANESCHI, Houston, TX (US); Riccardo MUZZIOLI, Houston, TX (US); Seth GAMMON, Houston, TX (US); David PIWNICA-WORMS, Houston, TX (US)

(73) Assignee: BOARD OF REGENTS, THE UNIVERSITY OF TEXAS SYSTEM, Austin, TX (US)

(21) Appl. No.: 18/554,403

(22) PCT Filed: Apr. 7, 2022
(86) PCT No.: PCT/US2022/023904

§ 371 Date: Oct. 6, 2023

Related U.S. Application Data
(60) Provisional application No. 63/172,181, filed on Apr. 8, 2021.

(51) Int. Cl. C07D 471/06; A61K 51/04

(52) U.S. Cl. CPC C07D 471/06; A61K 51/0459

(57) ABSTRACT

The synthesis and use of radiolabeled derivatives of poly-(ADP-ribose) (PARP) inhibitors in positron emission tomography (PET) imaging methods are disclosed. A novel non-radioactive analogue of the PARP inhibitor Talazoparib (TZ) provides a branch point for the syntheses of various radiolabeled Talazoparib derivatives. Aspects of the disclosure include such radiolabeled derivatives and TZ analogues, along with methods of use for diagnosis, treatment, imaging, and theranosis of cancer.

### CROSS REFERENCE TO RELATED APPLICATIONS

This application claims benefit of priority to U.S. Provisional Application No. 63/172,181, filed Apr. 8, 2021, which is hereby incorporated by reference in its entirety.

### BACKGROUND

### I. Field of the Invention

The present invention relates generally to the fields of biology, chemistry, and medicine, and more particularly relates to the fields of organic chemistry and nuclear medicine.

### II. Background

Poly-(ADP-ribose) polymerases (PARPs) are an enzyme family that catalyze the transfer of ADP-ribose from nicotinamide adenine dinucleotide (NAD+) to an acceptor protein. PARP family members play fundamental roles in single-strand DNA break (SSB) repair, cell signaling of DNA damage and inflammation, cell death, and cellular replication. In the cell nucleus, in particular, PARP1 and PARP2 (PARP1/2) detect and initiate an immediate cellular response to SSBs by signaling the enzymatic machinery involved in SSB repair. In selected cancer cells having mutations in the tumor suppressor BRCA1/2 or the total absence of BRCA1/2 (proteins crucial for the double strand DNA repair pathway), inhibition of PARP1/2 will result in either failure to repair the DNA break or force use of a more error-prone DNA repair system. This combination of deficiencies may increase genome instability, leading to cell death in cancer cells, a phenomena known as synthetic lethality, which can be exploited as a therapeutic strategy.

Studies have confirmed that cell lines that are deficient in BRCA1/2 or that present with mutations of these proteins are particularly sensitive to the inhibition of PARP. Various PARP inhibitors have been developed for oncological therapy, and PARP1 inhibitors are currently used for breast cancer and ovarian cancer treatment. Numerous PARP1 inhibitors also are used in the therapy of tumors with a low incidence of BRCA mutations, such as pancreatic and prostate cancer.

Imaging of PARP using a radiolabeled inhibitor has been proposed for patient selection, therapeutic dose optimization, and imaging target engagement of novel PARP-targeting agents. Several PARP imaging agents have been developed and studied as possible radiotracers. Most of these imaging tracers were developed by modifying the structure of an existing PARP inhibitor drug to accommodate an imaging isotope. Structurally-modified PARP inhibitors do not necessarily exhibit the same pharmacokinetic, pharmacodynamic, and therapeutic effects of the corresponding un-modified drugs. These differences in pharmacological properties can diminish the value of PET-based diagnoses. There is a need in the field of nuclear medicine for effective PET imaging agents that share the same structure as their unlabeled, well-established counterparts.

### SUMMARY

The present inventors have discovered a method for synthesizing a radiolabeled PARP inhibitor that retains the same chemical structure as its unlabeled counterpart. The method employs a copper-mediated ¹⁸F-radiofluorination strategy of a novel aryl boronic ester derivative of the PARP inhibitor Talazoparib, a Food and Drug Administration (FDA) approved PARP inhibitor. This novel derivative, also referred to herein as a “precursor.” provides access to ¹⁸F-Talazoparib (¹⁸F-TZ), the ¹⁸F-radiolabeled form of the structurally identical Talazoparib. This precursor also provides access to Talazoparib derivatives in which the phenyl group's fluorine moiety is replaced with a different halogen atom. These halogen-variants are bioisosteres of the parent Talazaparib compound that exhibit similar pharmacological properties.

Prediction of drug distribution and target engagement is a powerful tool to predict treatment response. The use of ¹⁸F-Talazoparib for PET imaging allows direct prediction of therapeutic Talazoparib distribution in tumors and tissues, given the structural identity with the non-radiolabeled drug, and thus, preserving all pharmacodynamic and pharmacokinetic properties of the compound, a distinct advantage for clinical translation and data interpretation. The present disclosure provides a means by which additional, closely-related compounds labeled with therapeutic radioisotopes can be synthesized. These radiolabeled compounds can be employed in radiotherapeutic, radioimmunotherapeutic, and theranostic applications.

Embodiments of the disclosure include methods for theranosis, methods for diagnosis, methods for treatment, methods for synthesis, compounds, and pharmaceutical compositions. Compounds of the present disclosure may include at least 1, 2, 3, or more of the following components: a boronate ester, a halogen, a halogen radioisotope, and an amine protecting group. One of more of these components may be excluded from certain embodiments. Methods of the present disclosure may include at least 1, 2, 3, or more of the following steps: diagnosing a patient having cancer, treating a patient having cancer, administering a PARP inhibitor to a subject, detecting a BRCA1 mutation in a subject, detecting a BRCA2 mutation in a subject, detecting a compound in a subject using an imaging technique, performing an imaging technique, and synthesizing a radiolabeled Talazoparib derivative. One of more of these steps may be excluded from certain embodiments.

Some embodiments of the disclosure are directed to an imaging method comprising administering to a subject a compound of formula (I):

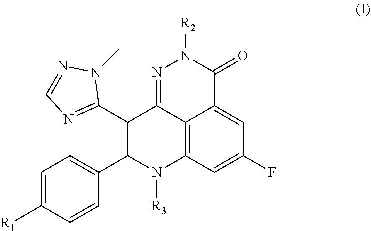

wherein R₁ is a halogen radioisotope, R₂ and R₃ are hydrogen, or a pharmaceutically acceptable salt thereof, and detecting the compound in a subject using an imaging technique. In some embodiments, the halogen radioisotope is selected from the group consisting of ¹⁸F, ⁷⁶Br, ⁷⁷Br, ¹²³I, ¹²⁴I, ¹²⁵I, ¹³¹I, and ²¹¹At. In some embodiments, R₁ is ¹⁸F. In some embodiments, R₁ is ⁷⁶Br. In some embodiments, R₁ is ⁷⁷Br. In some embodiments, R₁ is ¹²³I. In some embodiments, R₁ is ¹²⁴I. In some embodiments, R₁ is ¹²⁵I, In some embodiments, R₁ is ¹³¹I. In some embodiments, R₁ is ²¹¹At. Any one or more of these radioisotopes may be excluded from certain embodiments of the disclosure.

In some embodiments, the imaging technique selected from the group consisting of Positron Emission Tomography (PET), PET-Time-Activity Curve (TAC), PET-Magnetic Resonance Imaging (PET/MRI), PET/Computed Tomography (PET/CT), single photon emission computed tomography (SPECT), and SPECT/Computed Tomography (SPECT/CT). In some embodiments, the imaging method further comprises quantifying an amount of the compound in the subject. In some embodiments, the imaging method is used to obtain pharmacokinetic data of the compound. In some embodiments, the imaging method is used to obtain pharmacodynamic data of the compound. In some embodiments, the imaging method is used to evaluate PARP1/2 functional activity. In some embodiments, the method is used to predict drug distribution in the subject. In some embodiments, the imaging method is used to predict PARP inhibitor responsiveness. In some embodiments, the subject has been diagnosed with cancer. In some embodiments, the cancer is breast cancer. In some embodiments, the cancer is ovarian cancer. In some embodiments, the subject has at least one mutation in the BRCA1 or BRCA2 genes.

Some embodiments of the disclosure are directed to a compound of formula (I)

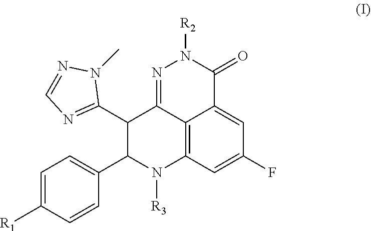

wherein R₁ is B(OH)₂, a boronate ester, a trifluoroborate, a halogen, or a radioisotope thereof, R₂ and R₃ are independently selected from hydrogen and an amine protecting group, or a pharmaceutically acceptable salt thereof, wherein when R₂ and R₃ are H, R₁ is not ¹⁹F. In some embodiments, the halogen radioisotope is selected from the group consisting of ¹⁸F, ⁷⁶Br, ⁷⁷Br, ¹²³I, ¹²⁴I, ¹²⁵I, ¹³¹I, and ²¹¹At. In some embodiments, R₁ is ¹⁸F. In some embodiments, R₁ is ¹⁶Br. In some embodiments, R₁ is ⁷⁷Br. In some embodiments, R₁ is ¹²³I. In some embodiments, R₁ is ¹²⁴I. In some embodiments, R₁ is ¹²⁵I. In some embodiments, R₁ is ¹³¹I. In some embodiments, R₁ is ²¹¹At. Any one or more of these radioisotopes may be excluded from certain embodiments of the disclosure.

In some embodiments, the amine protecting group is selected from the group consisting of Fmoc, BOC, acetyl, trifluoroacetamide, benzyl, p-methoxyphenyl benzoyl, methoxybenzyl, 3,4-dimethoxybenzyl, carboxybenzyl (Cbz), trityl, tosyl (p-toluenesulfonamide), Troc (trichloroethyl chloroformate), Nosyl (4-Nitrobenzenesulfonyl chloride), or a protecting group derived from a chloroalkyl ether selected from the group consisting of benzyl chloromethyl ether, chloromethyl methyl ether, tert-butyl chloromethyl ether, and methoxyethyl chloromethyl ether. The boronate ester may be any boronate ester known to those of skill in the art, including but not limited to the boronate ester groups disclosed in Chen et al., Advanced Synthesis & Catalysis 2020, 362, p. 3311-3331; and Thomas et al., Journal of the American Chemical Society 2018, 140, p. 4401-4416. Additional boronate esters include commercially-available boronate esters such as those sold by Sigma Aldrich. In some embodiments, the boronate ester is selected from the group consisting of pinacol boronate, 1,3-propanediol ester, catechol ester, neupentil glycol ester, dibutyl ester, N,N,N′,N′-tetramethyl-D-tartaric acid diamide ester, phenylboronic acid N-butyldiethanolamine ester, and hexylene glycolato)boron ester (Bhg). In some embodiments, the trifluoroborate is BF₃K. In some embodiments, a compound of formula (I) is further defined as one of:

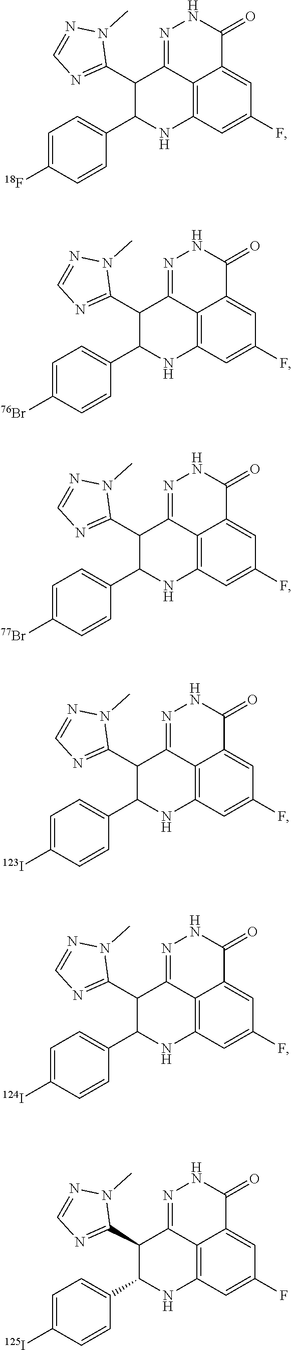 [SMILES: Cn1ncnc1C1c2n[nH]c(=O)c3cc(F)cc(c23)NC1c1ccc([18F])cc1 ; Cn1ncnc1C1c2n[nH]c(=O)c3cc(F)cc(c23)NC1c1ccc([76Br])cc1 ; Cn1ncnc1C1c2n[nH]c(=O)c3cc(F)cc(c23)NC1c1ccc([77Br])cc1 ; Cn1ncnc1C1c2n[nH]c(=O)c3cc(F)cc(c23)NC1c1ccc([123I])cc1 ; Cn1ncnc1C1c2n[nH]c(=O)c3cc(F)cc(c23)NC1c1ccc([124I])cc1 ; Cn1ncnc1[C@H]1c2n[nH]c(=O)c3cc(F)cc(c23)N[C@@H]1c1ccc([125I])cc1]

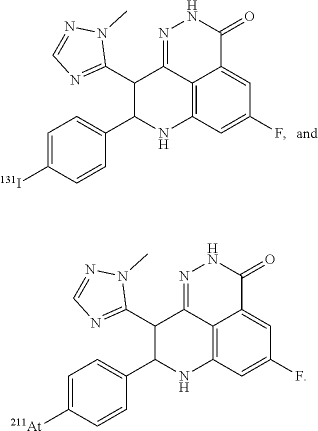

In some embodiments, a compound of formula (I) is defined as

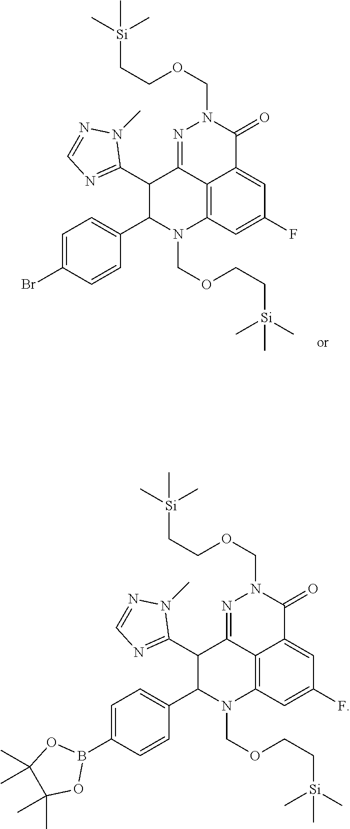

Some embodiments of the disclosure are directed to a method for producing a radiolabeled Talazoparib derivative, or a pharmaceutically acceptable salt thereof. In some embodiments, a method for producing a radiolabeled Talazoparib derivative comprises the steps of providing methyl-2-(4-bromophenyl)-7-fluoro-3-(1-methyl-1H-1,2,4-triazol-5-yl)-4-oxo-1,2,3,4-tetrahydro-quinoline-5-carboxylate; protecting the pyridazinone α-amine and the piperidine amine with amine protecting groups; substituting the phenyl 4-bromo group with a boronic acid or a boronate ester; substituting the 4-boronic acid or 4-boronate ester with a halogen radioisotope; and removing the amine protecting groups to provide the radiolabeled Talazoparib derivative.

In some embodiments, the radiolabeled Talazoparib derivative comprises a halogen radioisotope at the 4-phenyl position. In some embodiments, the amine protecting group is selected from the group consisting of Fmoc, BOC, acetyl, trifluoroacetamide, benzyl, p-methoxyphenyl benzoyl, methoxybenzyl, 3,4-dimethoxybenzyl, carboxybenzyl (Cbz), trityl, tosyl (p-toluenesulfonamide), Troc (trichloroethyl chloroformate), Nosyl (4-Nitrobenzenesulfonyl chloride), or a protecting group derived from a chloroalkyl ether selected from the group consisting of benzyl chloromethyl ether, chloromethyl methyl ether, tert-butyl chloromethyl ether, and methoxyethyl chloromethyl ether. In some embodiments, the boronate ester is pinacol boronate.

Some embodiments of the disclosure are directed to a method for diagnosing and treating a patient having cancer, comprising administering to a subject a compound of Formula (I):

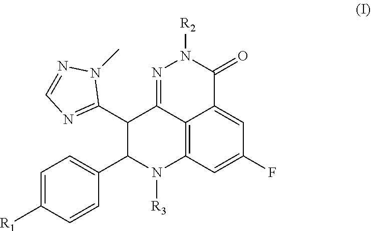

wherein R₁ is ¹⁸F, ⁷⁶Br, ⁷⁷Br, ¹²³I, ¹²⁴I, ¹²⁵I, ¹³¹I, or ²¹¹At and R₂ and R₃ are hydrogen, or a pharmaceutically acceptable salt thereof, and detecting the compound in the subject using an imaging technique. In some embodiments, R₁ is ¹⁸F. In some embodiments, R₁ is ⁷⁶Br. In some embodiments, R₁ is ⁷⁷Br. In some embodiments, R₁ is ¹²³I. In some embodiments, R₁ is ¹²⁴I. In some embodiments, R₁ is ¹²⁵I. In some embodiments, R₁ is ¹³¹I. In some embodiments, R₁ is ²¹¹At. Any one or more of these radioisotopes may be excluded from certain embodiments of the disclosure.

In some embodiments, the compound inhibits PARP1/2 activity. In some embodiments, the imaging technique selected from the group consisting of Positron Emission Tomography (PET), PET-Time-Activity Curve (TAC), PET-Magnetic Resonance Imaging (PET/MRI), PET/Computed Tomography (PET/CT), single photon emission computed tomography (SPECT), and SPECT/Computed Tomography (SPECT/CT). In some embodiments, the method further comprises quantifying an amount of the compound in the subject. In some embodiments, the method is used to obtain pharmacokinetic data of the compound. In some embodiments, the method is used to obtain pharmacodynamic data of the compound. In some embodiments, the method is used to monitor chemotherapy response in the subject. In some embodiments, the cancer is breast cancer. In some embodiments, the cancer is ovarian cancer. In some embodiments, the subject has at least one mutation in BRCA1 or BRCA2.

In some embodiments, the compound is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, or any combination thereof. In some embodiments, the administration is done prior to, concurrently with, or subsequent to an immunotherapeutic treatment. In some embodiments, the immunotherapeutic treatment is selected from the group consisting of an immune checkpoint inhibitor, T-cell transfer therapy, an immune system modulator, a monoclonal antibody, and a treatment vaccine. In some embodiments, administration of the compound affects at least one of cell cycle regulation, apoptosis, cell growth, and cell differentiation.

The “numerical values” and “ranges” provided for the various substituents are intended to encompass all integers within the recited range. For example, when defining n as an integer representing a value including from 1 to 100, where the value typically encompasses the integer specified as n±10% (or for smaller integers from 1 to 25, ±3), it should be understood that n can be an integer from 1 to 100 (e.g., 1, 2, 3, 4, 5, 6, 7, 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95, 99, 100, 105 or 110, or any between those listed).

Throughout this application, the term “about” is used to indicate that a value includes the inherent variation of error for the measurement or quantitation method.

The term “optional” or “optionally” means that the subsequently described event or circumstance may or may not occur, and that the description includes instances where said event or circumstance occurs and instances in which it does not.

As used herein, “pharmaceutically acceptable carrier” or “pharmaceutically acceptable excipient” includes any and all solvents, dispersion media, coatings, antibacterial and antifungal agents, isotonic and absorption delaying agents and the like. In several embodiments, these media and agents can be used in combination with pharmaceutically active substances. Except insofar as any conventional media or agent is incompatible with the active ingredient, its use in the therapeutic compositions is contemplated. Supplementary active ingredients can also be incorporated into the compositions.

The term “treatment” or “treating” means any treatment of a disease or disorder in a mammal, including: preventing or protecting against the disease or disorder, that is, causing the clinical symptoms not to develop; inhibiting the disease or disorder, that is, arresting or suppressing the development of clinical symptoms; and/or relieving the disease or disorder, that is, causing the regression of clinical symptoms.

The use of the word “a” or “an” when used in conjunction with the term “comprising” in the claims and/or the specification may mean “one.” but it is also consistent with the meaning of “one or more.” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

A “disease” is defined as a pathological condition of a body part, an organ, or a system resulting from any cause, such as infection, genetic defect, or environmental stress.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset.

The terms “inhibit,” “inhibiting,” and “inhibition,” (and grammatical equivalents) are used according to their plain and ordinary meaning in the area of medicine and biology. In the context of a physiological phenomenon, e.g., a symptom, in an untreated subject relative to a treated subject, these terms mean to limit, prevent, or block a biological/chemical reaction to achieve a reduction in the quantity and/or magnitude of the physiological phenomena in the treated subject as compared to a differentially treated subject (such as an untreated subject or a subject treated with a different dosage or mode of administration) by any amount that is detectable and/or recognized as clinically relevant by any medically trained personnel. In some embodiments, the quantity and/or magnitude of the physiological phenomena in the treated subject is about, at least about, or at most about 5, 10, 15, 20, 25, 30, 35, 40, 45, 50, 55, 60, 65, 70, 75, 80, 85, 90, 95, or 100% (or any range derivable therein) lower than the quantity and/or magnitude of the physiological phenomena in the differentially treated subject. Alternatively, in other embodiments, the quantity and/or magnitude of the physiological phenomena in the treated subject is about, at least about, or at most about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0 times (or any range derivable therein) lower than the quantity and/or magnitude of the physiological phenomena in the differentially treated subject.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect. All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Some aspects of the disclosure are directed towards the use of a composition as disclosed herein in any method disclosed herein. Some embodiments provide for the use of any composition disclosed herein for treating cancer. It is specifically contemplated that any step or element of an embodiment may be implemented in the context of any other step(s) or element(s) of a different embodiment disclosed herein.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Chemical structures of existing PET radiopharmaceuticals for imaging PARP1 activity in vivo.

FIG. 2 Schemes for syntheses of ⁷⁶Br-, ⁷⁷Br-, and ¹⁸F-labeled Talazoparib derivatives.

FIG. 3 Schemes for syntheses of organostannane Talazoparib derivatives.

FIGS. 4A-4B Talazoparib derivatives. FIG. 4A Talazoparib derivatives labeled with radioactive Iodine (¹²³I, ¹²⁴I, and ¹³¹I) and Astatine (²¹¹At). FIG. 4B Organostannane intermediate that can be used to synthesize Talazoparib and analogs thereof.

FIGS. 5A-5B Analytical radio-HPLC of ¹⁸F-Talazoparib, spiked with the reference compound, on an XBridge 4.6×250 mm, 3.5 μm C18 column. FIG. 5A Radioactive trace (gamma detector) FIG. 5B UV trace (254 nm).

FIGS. 6A-6B Analytical radio-HPLC of ¹⁸F-Talazoparib on an XBridge 4.6×250 mm. 3.5 μm C18 column. FIG. 6A Radioactive trace (gamma detector). FIG. 6B UV trace (254 nm).

FIG. 7 Inhibition titration curve of PARP1 activity by bromo-Talazoparib. The racemic mixture of bromo-Talazoparib preserved the ability to inhibited PARP1 (IC₅₀, 2.33 nM). Each data point represents the mean of three replicates at each concentration of the compound.

FIG. 8 ¹⁸F-Talazoparib uptake by MCF-7 cells under various conditions. Uptake at 37° C. (square), plus 100 nM of non-radioactive Talazoparib (diamond), with LY-335979 (triangle), with CP-100356 (open circle), or at 4° C. (filled circle) are shown. Data are means±standard deviations and statistical significance was confirmed using a 2-way ANOVA (**** p<0.0001).

FIGS. 9A-9B Distribution of ¹⁸F-Talazoparib in vivo. PET imaging after 120 minutes post injection of ¹⁸F-Talazoparib (LT, lymphoid tissue/salivary glands; L, liver). FIG. 9A PET imaging 2 hrs post i.v. injection of ¹⁸F-Talazoparib. FIG. 9B PET imaging showing blockade of target engagement of PARP1 with the pre-administration of 0.3 mg/kg cold Talazoparib 60 minutes before i.v. injection of ¹⁸F-Talazoparib.

FIGS. 10A-10B Quantification of ¹⁸F-Talazoparib blocking studies. FIG. 10A Target organ-to-non-target organ retention ratios in the control and blocking studies. FIG. 10B Biodistribution of ¹⁸F-Talazoparib at 2 hr post injection of vehicle-treated mice.

FIG. 11 shows a scheme for synthesis of a ⁷⁷Br-labeled Talazoparib derivative.

FIGS. 12A and 12B show results from analytical HPLC of ⁷⁷Br-Talazoparib analog on a XBridge 4.6×250 mm, 3.5 μm C18 column; elution profile of ⁷⁷Br-Talazoparib analog as spike (FIG. 12A) with the presence of cold Br-Talazoparib analog as reference compound (FIG. 12B). Red: gamma counter trace; Green: UV channel (wavelength: 254 nm).

FIG. 13 shows an inhibition titration curve of PARP1 activity by iodo-Talazoparib. The racemic mixture of iodo-Talazoparib preserved the ability to inhibit PARP1 (IC₅₀, 98.3 nM). Each data point represents the mean of three replicates at each concentration of the compound

### DETAILED DESCRIPTION OF THE INVENTION

Aspects of the present disclosure describe the synthesis and evaluation of novel PET radiopharmaceuticals. Radiolabeled Talazoparib analogs, as well as ¹⁸F-Talazoparib (¹⁸F-TZ), a radiolabeled variant that retains the parent drug structure, were synthesized and used to assess PARP expression/activation in vivo by PET imaging. The novel radioactive PET imaging agents were tested in several cancer cell lines that have different expression levels of PARP1, as well as various mutations in BRCA1 to evaluate cell retention. The biodistribution of ¹⁸F-TZ in healthy mice was evaluated and used to confirm active PARP-mediated tumor uptake.

### A. Chemical Definitions

As used herein, a “small molecule” refers to an organic compound that is frequently synthesized via conventional organic chemistry methods (e.g., in a laboratory). Typically, a small molecule is characterized in that it contains several carbon-carbon bonds and has a molecular weight of less than 1500 grams/mole. In certain embodiments, small molecules are less than 1000 grams/mole. In certain embodiments, small molecules are less than 550 grams/mole. In certain embodiments, small molecules are between 200 and 550 grams/mole. In certain embodiments, small molecules exclude peptides (e.g., compounds comprising 2 or more amino acids joined by a peptidyl bond). In certain embodiments, small molecules exclude nucleic acids.

As used herein, the term “amino” means —NH₂; the term “nitro” means —NO₂; the term “halo” or “halogen” designates —F, —Cl, —Br or —I; the term “mercapto” means —SH; the term “cyano” means —CN; the term “azido” means —N₃; the term “silyl” means —SiH₃, and the term “hydroxy” means —OH. In certain embodiments, a halogen may be —Br or —I.

Compounds described herein may be prepared synthetically using conventional organic chemistry methods known to those of skill in the art and/or are commercially available (e.g., ChemBridge Co., San Diego, CA).

Embodiments are also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like. A salt may be a pharmaceutically acceptable salt, for example. Thus, pharmaceutically acceptable salts of compounds of the present invention are contemplated.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids such as p-toluene sulfonic acid, and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like.

Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

Derivatives of compounds of the present invention are also contemplated. In certain aspects, “derivative” refers to a chemically modified compound that still retains the desired effects of the compound prior to the chemical modification. Such derivatives may have the addition, removal, or substitution of one or more chemical moieties on the parent molecule. Non-limiting examples of the types modifications that can be made to the compounds and structures disclosed herein include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, heteroatoms such as N, S, or O can be substituted into the structure instead of a carbon atom.

Compounds employed in methods of the invention may contain one or more asymmetrically-substituted carbon or nitrogen atoms, and may be isolated in optically active or racemic form. Thus, all chiral, diasteromeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diasteromers. In some embodiments, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, isotopes of carbon include ¹³C and ¹⁴C, isotopes of fluorine include ¹⁸F and ¹⁹F, isotopes of bromine include ⁷⁶Br and ⁷⁷Br, isotopes of iodine include ¹²³I, ¹²⁴I, ¹²⁵I, ¹³¹I, and one isotope of astatine is ²¹¹At.

As noted above, compounds of the present invention may exist in prodrug form. As used herein, “prodrug” is intended to include any covalently bonded carriers which release the active parent drug or compounds that are metabolized in vivo to an active drug or other compounds employed in the methods of the invention in vivo when such prodrug is administered to a subject. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a subject, cleaves to form a free hydroxyl, free amino, or carboxylic acid, respectively. Other examples include, but are not limited to, acetate, formate, and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (2002), which is incorporated herein by reference.

### B. Pharmaceutical Formulations and Administration Thereof

### 1. Pharmaceutical Formulations and Routes of Administration

Pharmaceutical compositions are provided herein that comprise an effective amount of one or more substances and/or additional agents dissolved or dispersed in a pharmaceutically acceptable carrier. The phrases “pharmaceutically acceptable” or “pharmacologically acceptable” refer to molecular entities and compositions that do not produce an adverse, allergic or other untoward reaction when administered to an animal, such as, for example, a human, as appropriate. The preparation of a pharmaceutical composition that contains at least one substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The compounds of the invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, intramuscularly, systemically, subcutaneously, subconjunctival, intravesicularly, mucosally, intrapericardially, intraumbilically, intraocularly, orally, locally, via inhalation (e.g., aerosol inhalation), via injection, via infusion, via continuous infusion, via localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 1990).

The actual dosage amount of a composition administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of a compound described herein. In other embodiments, the compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The substance may be formulated into a composition in a free base, neutral or salt form. Pharmaceutically acceptable salts, include the acid addition salts, e.g., those formed with the free amino groups of a proteinaccous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine, or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. It may be preferable to include isotonic agents, such as, for example, sugars, sodium chloride or combinations thereof.

In other embodiments, one may use eye drops, nasal solutions or sprays, aerosols or inhalants. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in certain embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. In certain embodiments, carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, or both.

Additional formulations which are suitable for other modes of administration include suppositories. Suppositories are solid dosage forms of various weights and shapes, usually medicated, for insertion into the rectum, vagina, or urethra. After insertion, suppositories soften, melt or dissolve in the cavity fluids. In general, for suppositories, traditional carriers may include, for example, polyalkylene glycols, triglycerides, or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, certain methods of preparation may include vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein.

In particular embodiments, prolonged absorption of an injectable composition can be brought about by the use in the compositions of agents delaying absorption, such as, for example, aluminum monostearate, gelatin, or combinations thereof.

### 2. Combination Therapy

The compositions and related methods of the present invention, particularly administration of a Talazoparib or derivative thereof may also be used in combination with the administration of additional anti-cancer therapies (e.g., chemotherapies, radiotherapies, immunotherapies, etc.).

Compounds discussed herein may precede, be co-current with and/or follow the other agents by intervals ranging from minutes to weeks. In embodiments where the agents are applied separately to a cell, tissue or organism, one would generally ensure that a significant period of time did not expire between the time of each delivery, such that the agents would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the candidate substance. In other aspects, one or more Talazoparib derivatives may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to administering a different anti-cancer, anti-proliferative, or anti-metastatic therapeutic. In some embodiments, more than one course of therapy may be employed. It is contemplated that multiple courses may be implemented.

### C. PARP and PARP Inhibitors

Poly-(ADP-ribose) polymerases (PARP) are a family of enzymes that catalyze the transfer of ADP-ribose from nicotinamide adenine dinucleotide (NAD+) to an acceptor protein. Formation of this long poly-ADP-ribose (PAR) negatively charged chain on the acceptor protein or on the enzyme itself controls various cellular processes and modulates cell signaling. The PARP superfamily is composed of eighteen different proteins mostly located in the nucleus and characterized by the presence of a specific PARP domain at the C-terminus. PARP1, the most abundant of this superfamily, is highly expressed in several tissues, such as brain, endocrine tissue (thyroid and parathyroid), bone marrow, and lymphoid tissues (tonsil, lymph nodes and spleen).

PARP1 is a multi-domain protein with two N-terminal zinc finger domains mediating DNA interactions and binding, a nuclear localization domain, a BRCT auto-modification motif and the classical PARP signature domain at the C-terminus. PARP1 plays a crucial role in the base excision repair pathway (BER) for restoration of single strain DNA (ssDNA) damage. Recent studies demonstrate that PARP1 also plays a role in adaptive immunity and in inflammatory response by modulating the ability of dendritic cells to stimulate T cells, modifying the generation of Treg cells from CD4+ T cells, and in the function and development of B cells. PARP1 is also involved in functional aspects of the innate immune system, impacting neutrophils, macrophages, dendritic cells and natural killer cells. In inflammatory processes, PARP1 can activate different factors such as NF-κB, can recruit and modify the function of neutrophils, and can PARylate proteins that are crucial in the inflammatory response, such as high mobility group box 1 protein (HMGB1).

In the absence of a functional single strand damage repair system, single strand damage may evolve into double-strand damage, which requires other molecular mechanisms to be activated for repair, such as homologous recombination (HR) or the more error-prone non-homologous end-joining (NHEJ) pathways. Breast Cancer-1/2 (BRCA1/2) are DNA damage response proteins that play crucial roles in several processes related to DNA stability, including cell checkpoint control, chromatin remodeling, ubiquitination, and repair of double stand DNA (dsDNA) breaks via the HR pathway. In the presence of BRCA1/2 mutations or in the total absence of either protein, cells either fail to repair DNA defects or are forced to use the NHEJ pathway. This may increase genome instability and lead to cell death.

Studies have confirmed that cell lines that are deficient in BRCA1/2 or contain mutations of these proteins are sensitive to inhibition of PARP. This exposes a vulnerability in cancer cells involving the BRCA and PARP genes called synthetic lethality. Two genes are synthetic lethal when mutation or inactivation of either one is compatible with cell life, but inactivation or mutation of both at the same time results in cell death. Given the relationship between BRCA1/2 and PARP, and the presence of BRCA mutations in numerous types of tumors, the search for innovative PARP inhibitors has been an active area in medicinal chemistry and translation to cancer therapy in recent years. Of all the PARP inhibitors studied, (8S,9R)-5-fluoro-8-(4-fluorophenyl)-9-(1-methyl·1H·1,2,4-triazol-5-yl)-2,7,8,9-tetrahydro-3H-pyrido[4,3,2-de]phthalazin-3-one (Talazoparib, BMN 673) presents a benzamide moiety for interaction with the active binding of PARP1, and appears to be the most potent therapeutic. It is thought that the presence of two stereogenic centers increases the ability to bind to PARP1, moving the affinity of this inhibitor into the sub-nanomolar range in purified enzyme assays (IC₅₀=0.57 nM). While PARP inhibitor therapy has provided an alternative in the treatment of BRCA mutated tumors, many tumors become resistant to therapy through the development of numerous resistance mechanisms, such as recovery in BRCA1 function by secondary mutation, residual activity in BRCA1, downregulation of PARP1, and upregulation of ATP-binding cassette transporters, such as the MDR1 P-glycoprotein (Pgp) or breast cancer resistance protein (BCRP), which can export drugs out of the cell, thus lowering intracellular drug concentrations, and reducing efficacy.

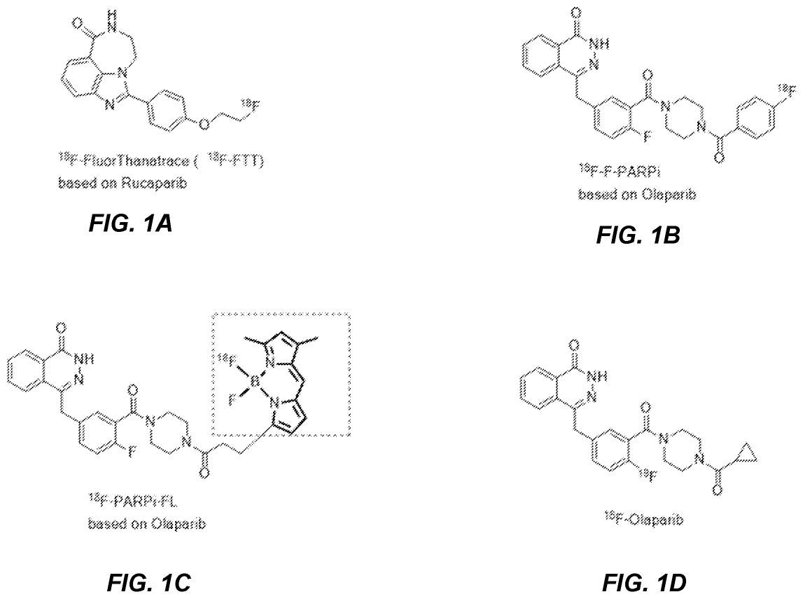

Positron Emission Tomography (PET) with a radiolabeled version of a PARP inhibitor has the potential to non-invasively provide insight on PARP1 expression/activation levels, PARP accessibility, and indirectly Pgp or BCRP functional transport activities. Several PARP imaging agents have been developed based on the structure of Olaparib (IC₅₀=3.6 nM) and Rucaparib (IC₅₀=4.2 nM) as building blocks. The molecular structure of ¹⁸F-FluorThanatracc (¹⁸F-FTT) (FIG. 1A) is based on the PARP1 inhibitor Rucaparib and was synthetized by nucleophilic substitution of the ethyl mesylate precursor. Rucaparib's original drug structure was modified by introducing a radiolabeled moiety, which allegedly has no detrimental effect on ¹⁸F-FTT's binding affinity, being that the nicotinamide-benzamide moiety is responsible for binding. The final compound maintains the ability to inhibit PARP1 (IC₅₀=6.3 nM) in purified enzymatic assays.

¹⁸F-FTT was the first PARP inhibitor imaging agent to be tested in humans (eight subjects with cancer and eight healthy volunteers) in a comparative study with murine models. In this first clinical study, subjects were imaged at different time points after tracer injection. PET imaging showed high uptake in spleen, pancreas, and liver, confirming a hepatobiliary execratory pathway. Lymph nodes were clearly visible in all the subjects. The main weakness of ¹⁸F-FTT is its metabolic instability. In mouse models, only 55% of the intact parent molecule was observed in the blood after 5 minutes and only 13% after 30 min. This high metabolic instability can confound PET imaging, since the image is based on tracking the radioisotope in whatever chemical form it exists and the radioactive fragments would not be distinguishable from intact parent agent.

The PARP inhibitor Olaparib was used as a template for the development of radiolabeled derivatives ¹⁸F-PARPi-FL and ¹⁸F-F-PARPi (FIGS. 1B and 1C). ¹⁸F-F-PARPi was obtained after conjugation of the 2H-phthalazin-1-one Olaparib fragment with 4-[¹⁸F]fluorobenzoic acid and the final compound still preserved the capacity of binding PARP1 (IC₅₀=2.83 nM). ¹⁸F-PARPi-FL contains the Olaparib core with a 4,4-difluoro-4-bora-3a,4a-diaza-s-indacene (BODIPY) fluorescent probe where one fluorine is replaced with ¹⁸F, providing a dual PET/fluorescent probe (FIG. 1C). The final compound preserved the binding ability towards PARP1 (IC₅₀=17.9 nM) in a purified enzyme assay. Intact ¹⁸F-Olaparib (FIG. 1D) was recently synthetized from a boronic ester precursor after a radiofluorination reaction and was the first PARP inhibitor-based PET tracer that represented the identical structure as the original therapeutic drug. However, the affinity of Olaparib towards PARP1 (IC₅₀=3.6 nM) is significantly lower than that of Talazoparib, which in principle can negatively impact signal-to-noise and PET image quality. Consequently, at tracer level, it is envisioned that the higher affinity of a Talazoparib-based PET tracer for PARP1 activity would provide superior signal-to-noise and contrast during PET imaging.

The imaging agents discussed above have been tested in vitro using various cell lines with different levels of PARP1 expression and in different animal models, rendering direct quantitative comparison difficult. Studies in vivo have shown an overall similar biodistribution with uptake in various organs such as spleen, liver, and pancreas. Modest uptake was present in kidneys, suggesting a mixed renal-hepatobiliary clearance pathway. Uptake was generally demonstrated to be blockable in the presence of excess non-radiative PARP inhibitor. Although the binding capacity towards PARP1 was maintained for many agents, modifications introduced to enable radiolabeling rendered them substantially different from the original drug structures. The key difference between the FDA-approved PARP1 inhibitors (Rucaparib, Olaparib and Talazoparib) lies in the plasma stability of these compounds. Talazoparib exhibits greater plasma stability (˜90 hours) than Rucaparib (˜17 hours) and Olaparib (˜14.9 hours) without the formation of secondary metabolites. Because Talazoparib remains the most potent inhibitor targeting PARP1, and given its greater plasma stability, a tracer that has the identical structure as Talazoparib is potentially superior to all the PET tracers discussed above. The new tracer presented herein represents a novel radiopharmaceutical compound to quantify PARP expression/activation in the tumor compartment using PET imaging. ¹⁸F-Talazoparib PET may also guide therapeutic choices by monitoring the functional activity of MDR1 Pgp and BCRP and their impact on drug resistance, and possibly provide data on the level of immune infiltration in the tumor compartment in vivo in real time by PET imaging.

### D. Therapeutic Methods

Aspects of the disclosure are directed to compositions and methods for therapeutic use. The compositions of the disclosure may be used for in vivo, in vitro, or ex vivo administration. The route of administration of the composition may be, for example, intratumoral, intravenous, intramuscular, intraperitoneal, subcutaneous, intraarticular, intrasynovial, intrathecal, oral, topical, through inhalation, or through a combination of two or more routes of administration.

### 1. Cancer Therapy

In some embodiments, the disclosed methods comprise administering a cancer therapy to a subject or patient. The cancer therapy may be chosen based on the expression level measurements, alone or in combination with the clinical risk score calculated for the subject. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be a checkpoint inhibitor therapy or an adoptive cell therapy. Any of these cancer therapies may also be excluded. Combinations of these therapies may also be administered.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pincaloma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; cosinophilic leukemia; monocytic leukemia; mast cell leukemia; megakaryoblastic leukemia; myeloid sarcoma; and hairy cell leukemia. In some embodiments, the cancer is breast cancer. In some embodiments, the cancer is HER2-negative breast cancer. In some embodiments, the cancer is BRCA1/2 mutant breast cancer. In some embodiments, the cancer is ovarian cancer.

In some embodiments, the cancer is a recurrent cancer. In some embodiments, the cancer is Stage I cancer. In some embodiments, the cancer is Stage II cancer. In some embodiments, the cancer is Stage III cancer. In some embodiments, the cancer is Stage IV cancer.

It is contemplated that a cancer treatment may exclude any of the cancer treatments described herein. Furthermore, embodiments of the disclosure include patients that have been previously treated with a therapy described herein, are currently being treated with a therapy described herein, or have not been treated with a therapy described herein. In some embodiments, the patient is one that has been determined to be resistant to a therapy described herein. In some embodiments, the patient is one that has been determined to be sensitive to a therapy described herein.

### 2. Cancer Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

a. Checkpoint Inhibitors and Combination Treatment

Embodiments of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

I. PD-1, PDL1, and PDL2 inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC. Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### II. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1. B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### III. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1, CDR2, and CDR3 domains of the VH region of an anti-LAG3 antibody, and the CDR1, CDR2 and CDR3 domains of the VL region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

### IV. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or VH and/or VL domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the VH region of an anti-TIM-3 antibody, and the CDR1, CDR2 and CDR3 domains of the VL region of an anti-TIM-3 antibody. In another embodiment, the antibody has at least about 70, 75, 80, 85, 90, 95, 97, or 99% (or any derivable range therein) variable region amino acid sequence identity with the above-mentioned antibodies.

b. Activation of Co-Stimulatory Molecules

In some embodiments, the immunotherapy comprises an agonist (also “activator”) of a co-stimulatory molecule. In some embodiments, the agonist comprises an activator of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include agonistic antibodies, polypeptides, compounds, and nucleic acids.

c. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

d. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

e. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

f. Adoptive T-Cell Therapy

Adoptive T cell therapy (ACT) is a form of passive immunization by the transfusion of T-cells (adoptive cell transfer). They are found in blood and tissue and usually activate when they find foreign pathogens. In particular, they may activate when a T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, which may limit or prevent immune-mediated tumor death.

Multiple ways of producing and obtaining tumor targeted T cells have been developed. T-cells specific to a tumor antigen can be removed from a tumor sample (tumor-infiltrating lymphocytes, or “TILs”) or filtered from blood. Subsequent activation and culturing may be performed ex vivo, with the resulting cells administered to a subject. Activation can take place through gene therapy and/or or by exposing the T cells to tumor antigens.

### 3. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

### 4. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### 5. Neoantigens

In some embodiments, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### 6. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., ctoposide, teniposide), antibiotics (e.g., dactinomycin, daunorubicin, doxorubicin, bleomycin, plicamycin and mitoxanthrone), enzymes (e.g., L-asparaginasc), and biological response modifiers (e.g., Interferon-α), and (d) Miscellaneous Agents, such as platinum coordination complexes (e.g., cisplatin, carboplatin), substituted urcas (e.g., hydroxyurca), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluoruracil; 5-FU) and floxuridine (fluoride-oxyuridine; FudR). 5-FU may be administered to a subject in a dosage of anywhere between about 7.5 to about 1000 mg/m². Further, 5-FU dosing schedules may be for a variety of time periods, for example up to six weeks, or as determined by one of ordinary skill in the art to which this disclosure pertains.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### 7. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 8. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### E. Examples

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

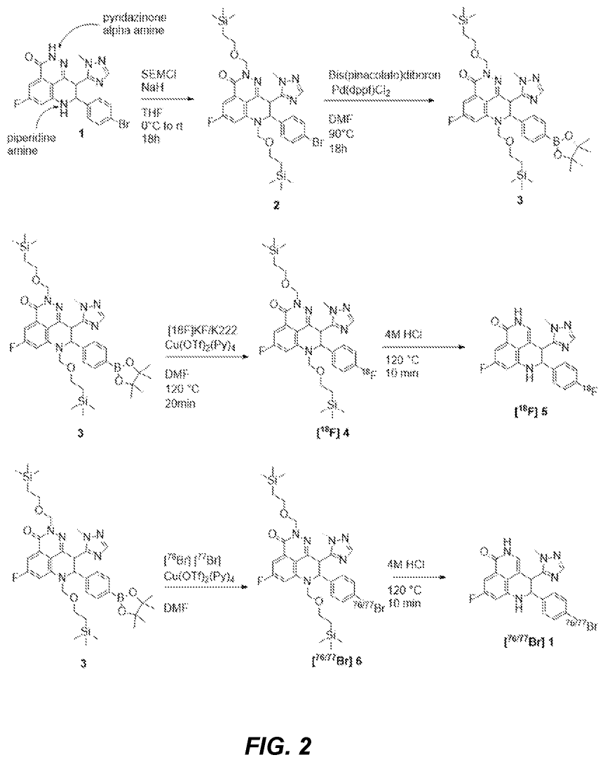

Schemes for the syntheses of representative compounds are provided in FIG. 2. The ^{76/77}Br annotation denotes that either the ⁷⁶Br or ⁷⁷Br isotope may be employed. The ^{123/124/131}I annotation denotes that either the ¹²³I, ¹²⁴I, or ¹³¹I isotope may be employed. The ^{123/124/125/131}I annotation denotes that either the ¹²³I, ¹²⁴I, ¹²⁵I, or ¹³¹I isotope may be employed. Combinations of isotopes are also contemplated. Therefore the methods and compositions disclosed herein can include ¹⁸F, ⁷⁶Br, ⁷⁷Br, ¹²³I, ¹²⁴I, ¹²⁵I, ¹³¹I, ²¹¹At, or any combination thereof.

### Example 1

### Synthesis of Compound 1

Synthesis of methyl 2-(4-bromophenyl)-7-fluoro-3-(1-methyl-1H-1,2,4-triazol-5-yl)-4-oxo-1,2,3,4-tetrahydroquinoline-5-carboxylate (1): Methyl-2-(4-bromophenyl)-7-fluoro-3-(1-methyl-1H-1,2,4-triazol-5-yl)-4-oxo-1,2,3,4-tetrahydro-quinoline-5-carboxylate (1) was synthesized using 4-bromobenzaldehyde in a methodology previously published (Wang et al., J Med Chem, 2016, 59(1): p. 335-57), the entirety of which is incorporated by reference. The product was obtained as a yellow powder of trans racemic mixtures [(8S,9R)/(8R,9S)]. ¹H NMR (500 MHZ, DMSO-d6) δ 12.36 (s, 1H), 7.81 (s, 1H), 7.54-7.52 (m, 2H), 7.43-7.41 (m, 2H), 7.09-7.07 (dd, J=2.5, 9.0 Hz 1H), 6.93-6.91 (dd, J=2.5, 8.9 Hz 1H), 5.01 (d, J=3.5 Hz 2H), 3.69 (s, 3H). ¹³C NMR (125 MHz, DMSO-d6) δ 166.50 (s), 164.39 (s), 159.37 (s), 152.32 (s), 150.79 (s), 148.85 (s), 141.39 (s), 138.99 (s), 131.78 (s), 130.76 (s), 121.95 (s), 111.78 (s), 103.29 (d), 99.02 (d), 59.10 (s), 42.77 (s), 35.39 (s). MS (ESI) m/z: 439.70 [M−H]⁻.

### Example 2

### Synthesis of Compound 2

Synthesis of 8-(4-bromophenyl)-5-fluoro-9-(1-methyl-1H-1,2,4-triazol-5-yl)-2,7-bis((2-(trimethylsilyl) ethoxy) methyl)-2,7,8,9-tetrahydro-3H-pyrido[4,3,2-de] phthalazin-3-one (2): This step involves protecting the pyridazinone α-amine and the piperidine amine with amine protecting groups. To a round bottom flask under argon atmosphere containing methyl 2-(4-bromophenyl)-7-fluoro-3-(1-methyl-1H-1,2,4-triazol-5-yl)-4-oxo-1,2,3,4-tetrahydroquinoline-5-carboxylate (412.9 mg, 0.94 mmol) at 0° C., in anhydrous THF (12 ml), sodium hydrate (180.5 mg. 7.52 mmol) was added portion-wise under argon atmosphere. The reaction was stirred for 45 min at 0° C. 2-(trimethylsilyl) ethoxymethyl chloride (SEM-Cl) was added dropwise over 25 min. The mixture was stirred overnight at room temperature. The reaction was quenched with water and extracted with DCM (4×35 ml). The organic layer was washed with brine (4×40 ml), then dried with Na₂SO₄ and the solvent was removed under vacuum. The crude material was purified using flash chromatography (% MeOH in DCM, 0% for 3CV. 0-20% in 15CV, 50% for 4CV) on a Biotage Isolera One system. 8-(4-bromophenyl)-5-fluoro-9-(1-methyl-1H-1,2,4-triazol-5-yl)-2.7-bis((2-(trimethylsilyl)ethoxy)methyl)-2,7,8,9-tetrahydro-3H-pyrido[4,3,2-de]phthalazin-3-one (2) was obtained as a yellow solid (536.1 mg. 82%). ¹H NMR (500 MHZ, chloroform-d) δ 7.73 (s, 1H), 7.46-7.44 (dd, J=2.3, 8.7 Hz 1H), 7.41-7.39 (m, 2H), 7.12-7.10 (dd, J=2.5, 11.0 Hz 1H), 7.07-7.05 (m, 2H), 5.43-5.36 (q. 2H), 5.21 (d. J=3.7 Hz, 1H.), 4.88-4.78 (q. 2H), 4.53 (d. J=4.0 Hz, 1H), 3.94 (s, 3H), 3.45-3.30 (m, 4H), 0.82 (t. J=8.17 Hz, 4H), 0.00018 (s. 18H). ¹³C NMR (125 MHZ, chloroform-d) δ 168.80 (s), 166.81 (s), 160.76 (d), 154.15 (s), 151.96 (s), 139.43 (s), 139.10 (s), 133.66 (d), 131.72 (s), 129.94 (d), 123.83 (s), 113.43 (s), 105.84 (d), 104.09 (d), 84.32 (s), 80.43 (s), 68.72 (s), 67.45 (s), 66.13 (s), 61.66 (s), 44.66 (s), 37.59 (s), 19.43 (d), 1.44 (s). MS (ESI) m/z: 701.71 [M+H]⁺.

### Example 3

### Synthesis of Compound 3

Synthesis of the final precursor 5-fluoro-9-(1-methyl-1H-1,2,4-triazol-5-yl)-8-(4-(4,4,5,5-tetramethyl-1,3,2-dioxaborolan-2-yl)phenyl)-2,7-bis((2-(trimethylsilyl)ethoxy)methyl)-2,7,8,9-tetrahydro-3H-pyrido[4,3,2-de]phthalazin-3-one (3): To an oven-dried round bottom flask under argon atmosphere, compound (2) (400 mg, 0.57 mmol), bis(pinacolato)diboron (327.6 mg, 1.3 mmol), Pd(dppf)Cl2 (14.6 mg. 0.02 mmol) and potassium acetate (176.7 mg, 1.8 mmol) were added. The flask was backfilled with argon and placed in a heating block at 90° C., upon which degassed DMF (3 ml) was added and the reaction was stirred overnight. The reaction mixture was passed through a celite plug, then diluted with water and extracted with EtOAc (4×30 ml). The organic layer was washed with saturated lithium chloride (3×40 ml) than dried over Na₂SO₄. Excess solvent was removed in vacuo, then DMF and methanol were added to precipitate the radiolabeling precursor 3 as a white solid (113 mg, 26.5%). ¹H NMR (500 MHZ, chloroform-d) δ 7.81 (s, 1H), 7.79-7.78 (d. J=8.0 Hz, 2H), 7.54-7.52 (dd, J=2.4 Hz, 8.3 Hz 1H), 7.26-7.25 (d, J=8.0 Hz, 2H), 7.22-7.20 (dd, J=2.2 Hz, 11.0 Hz 1H), 5.48-5.44 (q, 2H), 5.31 (d. J=4.3 Hz, 1H,), 4.95-4.80 (q, 2H), 4.46 (d. J=4.6 Hz, 1H), 3.98 (s, 3H), 3.68-3.65 (m, 2H), 3.49-3.37 (m, 2H), 1.40 (s, 12H), 0.88 (t, J=8.2 Hz, 4H), 0.00010 (s, 18H). ¹³C NMR (125 MHz, chloroform-d) δ 168.76 (s), 166.75 (s), 160.86 (d), 154.36 (s), 151.97 (s), 147.69 (d), 143.09 (s), 139.49 (s), 136.94 (s), 131.68 (s), 127.65 (s), 113.70 (s), 105.86 (d), 103.99 (d), 85.42 (s), 83.55 (s), 80.46 (s), 68.66 (s), 67.40 (s), 66.73 (s), 44.90 (s), 37.43 (s), 26.28 (s), 19.45 (d), 1.44 (s). MS (ESI) m/z: 771.83 [M+Na]⁺.

### Example 4

### Synthesis of Compound 5

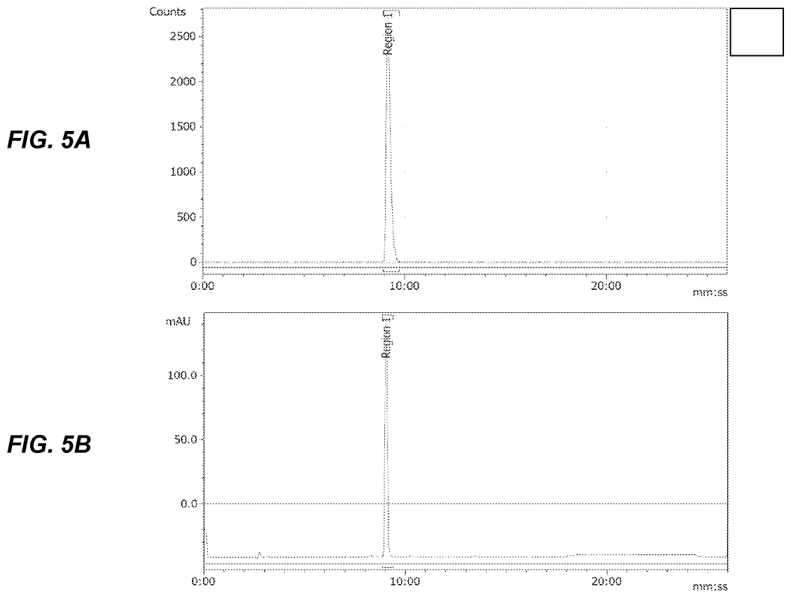

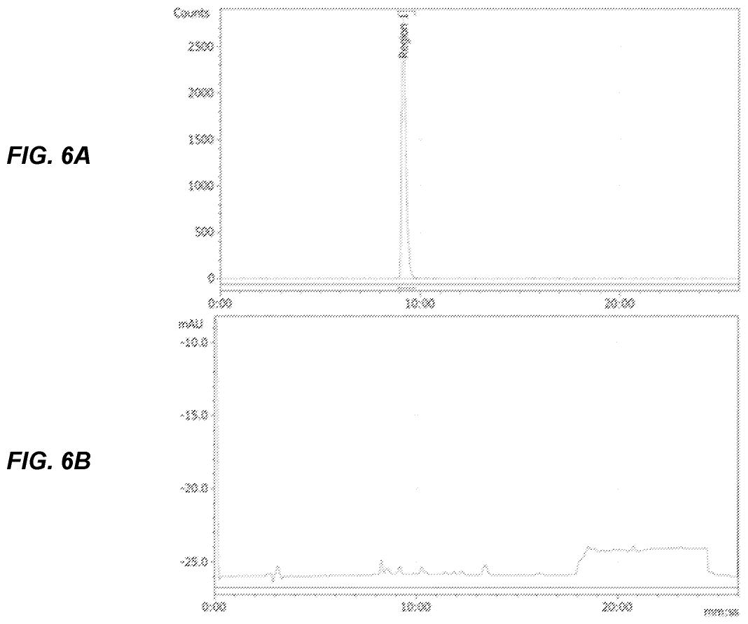

Synthesis of 5-[¹⁸F]fluoro-8-(4-fluorophenyl)-9-(1-methyl·1H·1,2,4-triazol-5-yl)-2,7,8,9-tetrahydro-3H-pyrido[4,3,2-de]phthalazin-3-one (Talazoparib, [¹⁸F]5): Radiosynthesis was performed on a TracerLab FX (General Electric Healthcare, Münster, Germany) automatic module. [¹⁸F]fluoride was obtained as an aqueous solution from the MD Anderson Cyclotron Radiochemical Facility (CRF). [¹⁸F]Fluoride was adsorbed on an ion exchange cartridge (pre-conditioned Sep-PAK® Light QMA Cartridge, ABX GmbH, Radeberg, Germany). [¹⁸F]fluoride was flushed into the reaction vial with a potassium carbonate and Kryptofix 2.2.2. water/CH₃CN solution (700 μL; 52.8 mg of K₂CO₃, 240.1 mg of K222, 4 mL of water, 16 mL of CH₃CN). The solution was dried under vacuum and under nitrogen flow at 60° C. for 2 min. 500 μL of dry CH₃CN was added and then the mixture was azeotropically dried at 120° C. for an additional 3 min. Synthesis of ¹⁸F-Talazoparib was first carried out by adding the N-silanyl-protected boronic pinacol ester precursor (3, 5-10 mg) and [Cu(OTf)₂(Py)₄] (15-17 mg) in dry DMF (600 μL) to the dried [¹⁸F]fluoride. Air was allowed to enter into the reactor by leaving vial 3 open during the setup of the instrument and closed before starting the synthesis. The mixture was stirred at 120° C. for 20 min, cooled down at 80° ° C. to generate intermediate [¹⁸F]4, and then HCl 4M (1 mL) was added to deprotect [¹⁸F]4, yielding crude product [¹⁸F]5. The mixture was stirred at 120° C. for 10 min, cooled down at 30° C. and diluted with water (3.1 mL). The crude was purified by semi preparative HPLC (Luna 5 μm C18(2) 100 Å, 250×10 mm) eluting with 30% MeCN/water (0.085% H₃PO₄) [rt=17 min]. For final dispensation, the desired radioactive product was collected into a TracerLab collection flask pre-filled with water (26 mL). The solution was loaded onto a Sep-Pak C18 Plus Light Cartridge, 130 mg Sorbent (Sep-PAK®, Waters, Milford, USA). Cartridges were washed with 8 mL of water, dried under nitrogen and eluted with ethanol (1 mL). The overall synthesis time was approximately 100 min. Activity was determined by dose calibrator and a sample taken for quality control (QC). QC was performed by analytical radio-HPLC (Agilent 1260 infinity II equipped with an in-line LabLogic flow HPLC radio detector) on a C18 column (Waters, XBridge C18 column, 4.6×250 mm, 3.5 μm), using a water (0.1% (v/v) TFA) and CH₃CN (0.1% (v/v) TFA) gradient (20% B→40% in 5 min, 40% B for 10 min, 40% B→95% B in 6 sec, 95% B for 6 min) at a flow rate of 1 mL/min. Under these conditions, ¹⁸F-Talazoparib presented a retention time of ˜9 minutes (FIG. 6A). The identity of the radiolabeled compound was confirmed by co-elution of the original cold standard (FIGS. 5A and 5B).

¹⁸F-Talazoparib was synthesized from precursor (3) in 3.64±0.81% (n=5) radiochemical yield. ¹⁸F-Talazoparib was obtained in >99% radiochemical purity and showed up to a 4 h shelf stability in PBS (10% EtOH). ¹⁸F-Talazoparib was also stable in full human plasma up to 4 h at 37° C.

### Example 5

### Synthesis of Compound 9

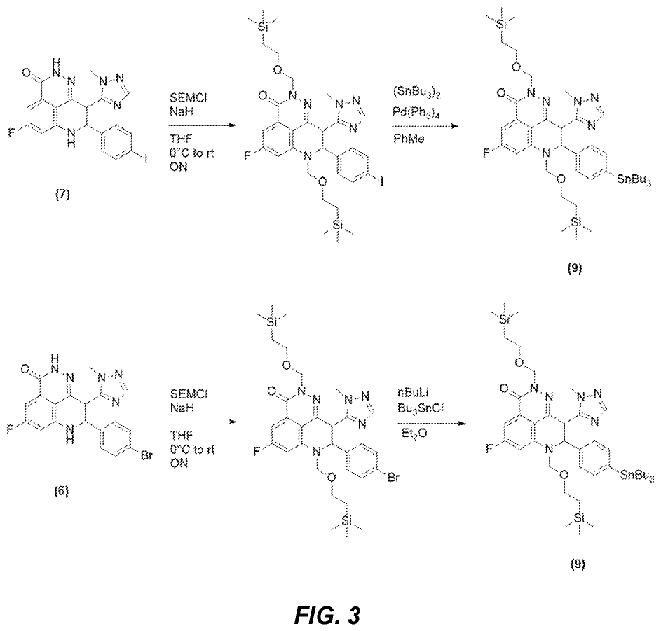

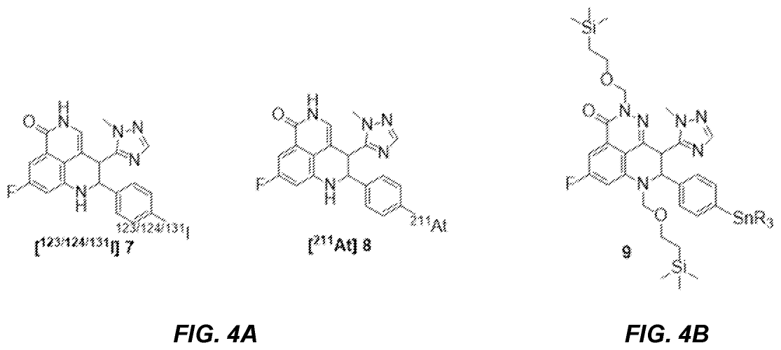

FIG. 3 depicts two schemes for the synthesis of organostannane compound 9, a precursor compound for Talazoparib variants. Compound 9 may be synthesized from the corresponding non-radioactive iodine derivative 7 by reaction with (R₃Sn)₂, where R is an alkyl group such as methyl, n-butyl, or phenyl or other organostannane substituent known to those of skill in the art. Alternatively, organostannane compound 9 may be synthesized from the corresponding bromine derivative 6 by reaction with R₃SnCl, via Grignard or by employing nBuLi to enable the transmetallation reaction. Other organostannane syntheses known to those of skill in the art may be employed, such as those that employ trimethylammonium salts or phosphoric esters analogues of iodide, or any of the methods disclosed in Mao et al., Journal of Organic Chemistry 2019, 84, p. 463-471 or Ghazi et al., Archives of Organic and Inorganic Chemical Sciences 2018, 3, p. 344-352, both of which are incorporated by reference in their entireties. Non-radioactive iodine derivatives 7 can be synthesized modifying the synthetic procedure used to synthesize 6 where p-iodobenzaldehyde replaces p-bromobenzaldehyde. Radioactive bromine-, iodine-, and astatine-containing Talazoparib variants (FIG. 4) can be synthesized from the corresponding boronic acid/ester, stannanes, by demetallation halogenation or, for radioactive iodine compound 7, by isotopic exchange as disclosed by Dubost et al., Journal of Organic Chemistry 2020, 85, p. 8300-8310, which is incorporated by reference in its entirety.

### Example 6

### Biochemical Characterization of Compound 1

The ability of the bromophenyl analog of Talazoparib, methyl 2-(4-bromophenyl)-7-fluoro-3-(1-methyl-1H-1,2,4-triazol-5-yl)-4-oxo-1,2,3,4-tetrahydroquinoline-5-carboxylate (1), to inhibit purified PARP1 enzyme activity was assessed using a PARP1 apoptosis assay kit (Trevigen, cat. no. 4685-096-K) following the manufacturer's instructions. Analysis was carried out in triplicate using a concentration range of inhibitor between 1,000 and 0.01 nM (1,000 nM, 100 nM, 10 nM, 1 nM, 0.1 nM and 0.01 nM); the IC₅₀ values were calculated using GraphPad Prism8 software (San Diego, CA, USA).

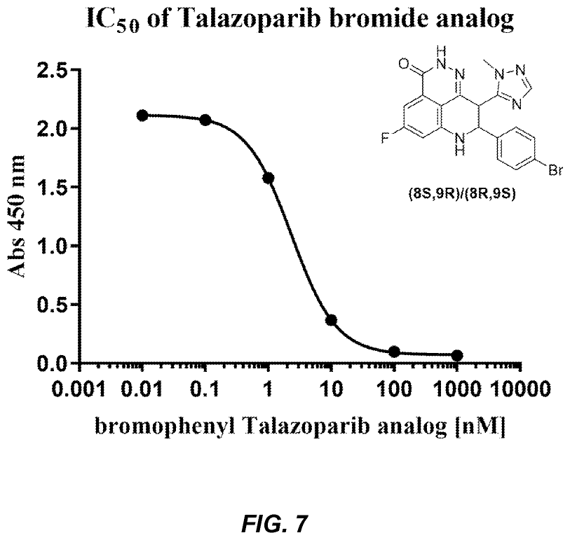

A racemic mixture [(8S,9R)/(8R,9S)] of the bromophenyl analog of Talazoparib, methyl 2-(4-bromophenyl)-7-fluoro-3-(1-methyl-1H-1,2,4-triazol-5-yl)-4-oxo-1,2,3,4-tetrahydroquinoline-5-carboxylate (1), was tested to evaluate its ability to inhibit PARP1. The inhibition titration curve obtained is shown in FIG. 7. Replacement of a fluorine atom with a bromine atom maintained the ability to inhibit PARP1 in purified enzyme assays with an IC₅₀ of 2.33 nM (as a racemic mixture), comparable to the reported IC₅₀ value of the racemic mixture of parental fluorophenyl-Talazoparib (2.14 nM).

### Example 7

### Cellular Uptake of ¹⁸F-Talazoparib

Cellular uptake, cold blocking, and MDR reversal of ¹⁸F-Talazoparib were studied in MCF-7 cell lines stably transfected with an expression cassette for MDR1 P-glycoprotein (Pgp) or the empty vector negative control. All experiments were performed in quadruplicate in a 24 well plate format. For cell uptake studies, 1×10⁵ MCF7 cells were incubate in MEBSS (1 ml), and then ¹⁸F-Talazoparib was added (12.5 kBq) to each well and incubated at 37° C. for 120 min. For blocking experiments, cells were preincubated for 30 min at 37° C. with excess non-radioactive Talazoparib (100 nM). In the MDR reversal experiments, LY-335979 trihydrochloride (LY), a Pgp inhibitor, or CP-100356 hydrochloride (CP), a mixed Pgp and BCRP inhibitor, was added and cells preincubated for 30 min at 37° C. Experiment that abrogated metabolism were performed at 4° C., a temperature at which cellular membranes become less fluid and permeability is low. After 120 min, an aliquot of the cell media (200 μl) was obtained for gamma counting, the remaining media removed, and cells washed with MEBSS. Cells were then lysed with 1% SDS, 10 mM sodium borate for 30 min at room temperature. Radioactivity contained within the aliquots of cell media and cell lysates were measured using a WIZARD² 2480 automatic gamma counter (ParkinElmer). Protein contents were quantified using BCA protein assay kit (Thermos Scientific) according to the manufacture's protocol using bovine serum albumin (BSA) as the protein standard. Cell uptake of ¹⁸F-Talazoparib were normalized to protein content and expressed as a tracer ratio ((cpm/mg protein)/(cpm/mL)).

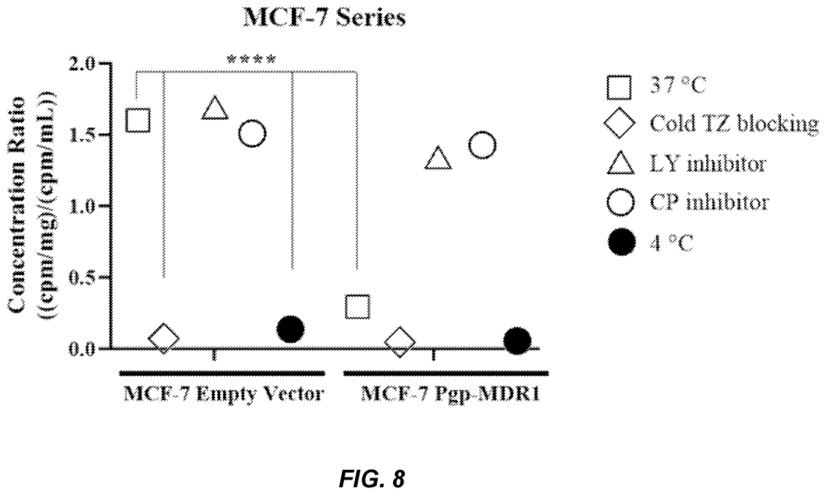

Cell uptake of radioactive ¹⁸F-Talazoparib in MCF-7 cells is shown in FIG. 8. After 120 min of incubation at 37° C. with ¹⁸F-Talazoparib, MCF-7 empty vector cells showed high uptake of the tracer compared to cells incubated at 4° C. Cold temperature conditions generally lower cell metabolism, and decrease membrane fluidity and solute permeability, thereby excluding non-targeted cell surface binding of the tracer as the mechanism of retention. As additional proof of targeting, cell uptake at 37° C. was completely blocked in the presence of excess non-radioactive Talazoparib. In MCF-7 empty vector cells, MDR inhibitors had no effect as expected. By contrast, when MDR1 Pgp was expressed in MCF-7 cells, the efflux of tracer induced a drastic decrease in the net retention of ¹⁸F-Talazoparib. This basal level was further reduced by 4° C. temperatures and fully displaced by excess cold Talazoparib. By contrast, when Pgp-mediated efflux of tracer was inhibited with LY or CP, cellular retention of ¹⁸F-Talazoparib was increased back to levels compared to MCF-7 cells lacking MDR1 Pgp, consistent with expectations that ¹⁸F-Talazoparib is recognized as a transport substrate by MDR1 Pgp.

### Example 8

### In Vivo Imaging

Preliminary PET images were acquired for 10 min using a 15 cm field of view (FOV); CT images were acquired for fusion using a 7 cm FOV and automatically “stitched” and fused to the PET imaging in the reconstruction software (Albira Suite, Bruker). For blocking experiments in vivo, mice were injected IV with 0.3 mg/kg of cold Talazoparib 60 minutes before injection of ¹⁸F-Talazoparib. Image data were decay corrected to injection time (Albira, Bruker) and expressed as % ID/cc or SUV as indicated (PMOD, PMOD Technologies). Actual injected dose was calculated based on measuring the pre- and post-injection activity in the syringe with a dose calibrator (Capintec) and mice were individually weighed to calculate individual standardized uptake values (SUV).

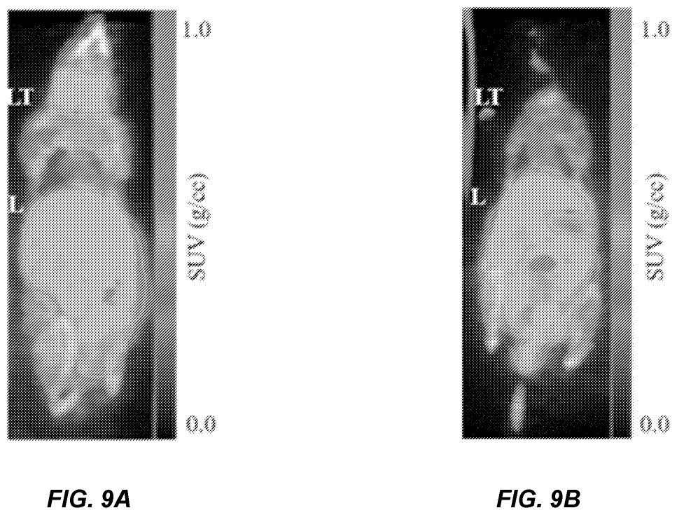

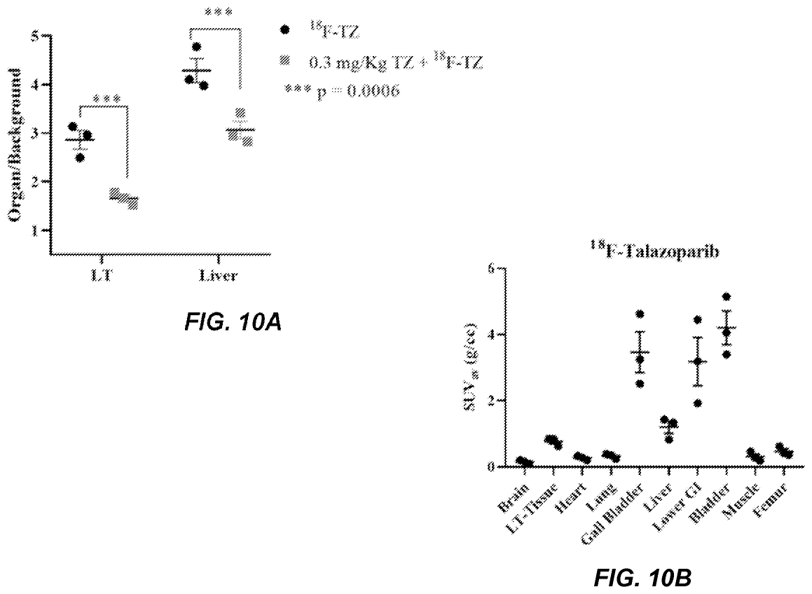

In vivo whole body biodistribution and blocking experiments were performed using a small animal PET/CT scanner after the injection of ¹⁸F-Talazoparib (˜100 μCi) in a nude female mouse; images were acquired two hours post injection and decay corrected to the injection time. Coronal images (FIG. 9A) showed uptake in the lymphoid tissue (LT) located at the base of the neck, in the liver (L), and in excretory organs, such as bladder and intestine. The lymphoid tissue and liver-specific uptake of ¹⁸F-Talazoparib could be blocked by excess cold Talazoparib (FIGS. 9B and 10A), indicating specific uptake of ¹⁸F-Talazoparib in these tissues. A mixed excretion pathway (renal and hepatobiliary clearance) was observed based upon quantification of the 2 hr static images of vehicle-treated nude mice (FIG. 10B).

### Example 9

### Synthesis and Analysis of ⁷⁷Br-Labeled Talazoparib

⁷⁷Br-labeled Talazoparib (“⁷⁷Br-Talazoparib”) was synthesized as shown in the synthesis scheme in FIG. 11. Analytical HPLC was performed on the resultant product, and compared to cold Br-Talazoparib as a reference. Results are shown in FIGS. 12A and 12B.

### Example 10

### Biochemical Characterization of Iodo-Talazoparib

The ability of the iodophenyl analog of Talazoparib, methyl 2-(4-iodophenyl)-7-fluoro-3-(1-methyl-1H-1,2,4-triazol-5-yl)-4-oxo-1,2,3,4-tetrahydroquinoline-5-carboxylate, to inhibit purified PARP1 enzyme activity was assessed using a PARP1 apoptosis assay kit (Trevigen, cat. no. 4685-096-K) following the manufacturer's instructions. Analysis was carried out in triplicate using a concentration range of inhibitor between 1,000 and 0.01 nM (1,000 nM, 100 nM, 10 nM, 1 nM, 0.1 nM and 0.01 nM); the IC₅₀ values were calculated using GraphPad Prism8 software (San Diego, CA, USA).

A racemic mixture of the iodophenyl analog of Talazoparib, methyl 2-(4-iodophenyl)-7-fluoro-3-(1-methyl-1H-1,2,4-triazol-5-yl)-4-oxo-1,2,3,4-tetrahydroquinoline-5-carboxylate, was tested to evaluate its ability to inhibit PARP1. The inhibition titration curve obtained is shown in FIG. 13. Replacement of a fluorine atom with an iodine atom maintained the ability to inhibit PARP1 in purified enzyme assays with an IC₅₀ of 98.3 nM (as a racemic mixture).

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A compound of Formula (I): 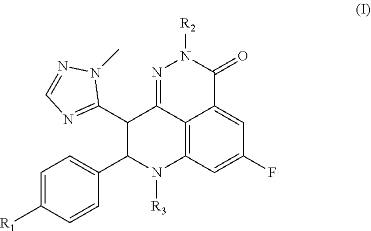
wherein:
R₁ is B(OH)₂, a boronate ester, a halogen, or a radioisotope thereof; and
R₂ and R₃ are independently selected from hydrogen, and an amine protecting group;
or a pharmaceutically acceptable salt thereof;
wherein when R₂ and R₃ are H, R₁ is not ¹⁹F.

2. The compound of claim 1, wherein R₁ is a halogen radioisotope selected from the group consisting of ¹⁸F, ⁷⁶Br, ⁷⁷Br, ¹²³I, ¹²⁴I, ¹²⁵I, ¹³¹I, and ²¹¹At.

3. The compound of claim 2, wherein R₁ is ¹⁸F.

4. The compound of claim 2, wherein R₁ is ⁷⁶Br.

5. The compound of claim 2, wherein R₁ is ⁷⁷Br.

6. The compound of any of claims 1-5, wherein the amine protecting group is selected from the group consisting of Fmoc, BOC, acetyl, trifluoroacetamide, benzyl, p-methoxyphenyl benzoyl, methoxybenzyl, 3,4-dimethoxybenzyl, carboxybenzyl (Cbz), trityl, tosyl (p-toluenesulfonamide), Troc (trichloroethyl chloroformate), and Nosyl (4-Nitrobenzenesulfonyl chloride), or is a protecting group derived from a chloroalkyl ether selected from the group consisting of benzyl chloromethyl ether, chloromethyl methyl ether, tert-butyl chloromethyl ether, and methoxyethyl chloromethyl ether.

7. The compound of claim 1, wherein R₁ is pinacol boronate.

8. The compound of any of claims 1-7, wherein the compound is further defined as one of:

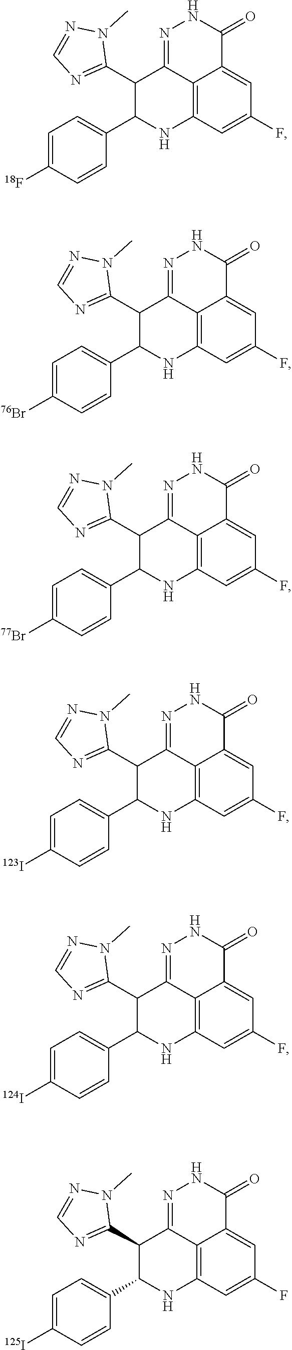 [SMILES: Cn1ncnc1C1c2n[nH]c(=O)c3cc(F)cc(c23)NC1c1ccc([18F])cc1 ; Cn1ncnc1C1c2n[nH]c(=O)c3cc(F)cc(c23)NC1c1ccc([76Br])cc1 ; Cn1ncnc1C1c2n[nH]c(=O)c3cc(F)cc(c23)NC1c1ccc([77Br])cc1 ; Cn1ncnc1C1c2n[nH]c(=O)c3cc(F)cc(c23)NC1c1ccc([123I])cc1 ; Cn1ncnc1C1c2n[nH]c(=O)c3cc(F)cc(c23)NC1c1ccc([124I])cc1 ; Cn1ncnc1[C@H]1c2n[nH]c(=O)c3cc(F)cc(c23)N[C@@H]1c1ccc([125I])cc1]

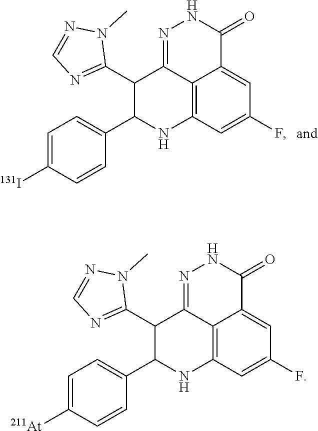

9. The compound of any of claims 1-7, wherein the compound is further defined as:

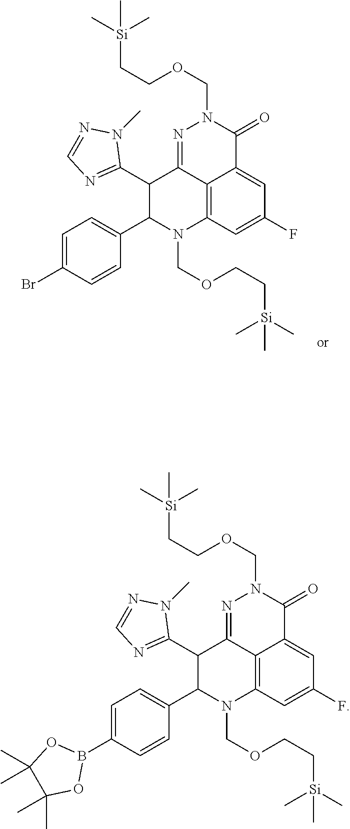

10. An imaging method comprising administering to a subject a compound of Formula (I): 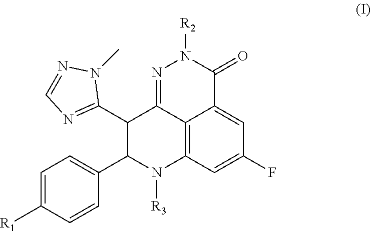
wherein:
R₁ is a halogen radioisotope; and
R₂ and R₃ are hydrogen;
or a pharmaceutically acceptable salt thereof;
and detecting the compound in the subject using an imaging technique.

11. The method of claim 10, wherein the halogen radioisotope is selected from the group consisting of ¹⁸F, ⁷⁶Br, ⁷⁷Br, ¹²³I, ¹²⁴I, ¹²⁵I, ¹³¹I, and ²¹¹At.

12. The method of claim 11, wherein R₁ is ¹⁸F.

13. The method of claim 11, wherein R₁ is ⁷⁶Br.

14. The method of claim 11, wherein R₁ is ⁷⁷Br.

15. The method of any of claims 10-14, wherein the imaging technique is selected from the group consisting of Positron Emission Tomography (PET), PET-Time-Activity Curve (TAC), PET-Magnetic Resonance Imaging (PET/MRI), PET/Computed Tomography (PET/CT), single photon emission computed tomography (SPECT), and SPECT/Computed Tomography (SPECT/CT).

16. The method of any of claims 10-15, further comprising quantifying an amount of the compound in the subject.

17. The method of any of claims 10-16, wherein the method is used to obtain pharmacokinetic data of the compound.

18. The method of any of claims 10-17, wherein the method is used to obtain pharmacodynamic data of the compound.

19. The method of any of claims 10-18, wherein the method is used to evaluate PARP1/2 functional activity.

20. The method of any of claims 10-19, wherein the method is used to predict PARP inhibitor responsiveness.

21. The method of any of claims 10-20, wherein the method is used to predict drug distribution in the subject.

22. The method of any of claims 10-21, wherein the subject is suspected of having cancer.

23. The method of any of claims 10-21, wherein the subject has cancer.

24. The method of claim 22 or 23, wherein the cancer is breast cancer or ovarian cancer.

25. A method for producing a radiolabeled Talazoparib derivative, comprising the steps of:
a) providing methyl-2-(4-bromophenyl)-7-fluoro-3-(1-methyl-1H-1,2,4-triazol-5-yl)-4-oxo-1,2,3,4-tetrahydro-quinoline-5-carboxylate;
b) protecting the pyridazinone α-amine and the piperidine amine with amine protecting groups;
c) substituting the phenyl 4-bromo group with a boronic acid or a boronate ester;
d) substituting the 4-boronic acid or 4-boronate ester with a halogen radioisotope; and
e) removing the amine protecting groups to provide the radiolabeled Talazoparib derivative.

26. The method of claim 25, wherein the radiolabeled Talazoparib derivative comprises a halogen radioisotope at the 4-phenyl position.

27. The method of claim 26, wherein the halogen radioisotope is selected from the group consisting of ¹⁸F, ⁷⁶Br, ⁷⁷Br, ¹²³I, ¹²⁴I, ¹²⁵I, ¹³¹I, and ²¹¹At.

28. The method of claim 27, wherein the halogen radioisotope is ¹⁸F.

29. The method of claim 27, wherein the halogen radioisotope is ⁷⁶Br.

30. The method of claim 27, wherein the halogen radioisotope is ⁷⁷Br.

31. The method of any of claims 25-30, wherein the amine protecting groups are selected from the group consisting of Fmoc, BOC, acetyl, trifluoroacetamide, benzyl, p-methoxyphenyl benzoyl, methoxybenzyl, 3,4-dimethoxybenzyl, carboxybenzyl (Cbz), trityl, tosyl (p-toluenesulfonamide), Troc (trichloroethyl chloroformate), Nosyl (4-Nitrobenzenesulfonyl chloride), or a protecting group derived from a chloroalkyl ether selected from the group consisting of benzyl chloromethyl ether, chloromethyl methyl ether, tert-butyl chloromethyl ether, and methoxyethyl chloromethyl ether.

32. The method of any of claims 25-31, wherein the boronate ester is pinacol boronate.

33. A theranostic method for diagnosing and treating a patient having cancer, comprising (a) administering to a subject a compound of Formula (I): 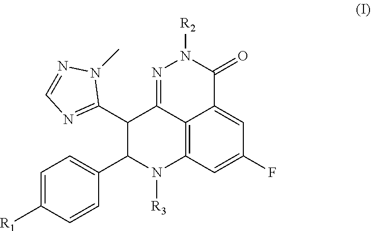
wherein:
R₁ is ¹⁸F, ⁷⁶Br, ⁷⁷Br, ¹²³I, ¹²⁴I, ¹²⁵I, ¹³¹I, or ²¹¹At; and
R₂ and R₃ are hydrogen;
or a pharmaceutically acceptable salt thereof;
and (b) detecting the compound in the subject using an imaging technique.

34. The method of claim 33, wherein R₁ is ¹⁸F.

35. The method of claim 33, wherein R₁ is ⁷⁶Br.

36. The method of claim 33, wherein R₁ is ⁷⁷Br.

37. The method of any of claims 33-36, wherein the compound inhibits PARP1/2 activity.

38. The method of any of claims 33-37, wherein the imaging technique selected from the group consisting of Positron Emission Tomography (PET), PET-Time-Activity Curve (TAC), PET-Magnetic Resonance Imaging (PET/MRI), PET/Computed Tomography (PET/CT), single photon emission computed tomography (SPECT), and SPECT/Computed Tomography (SPECT/CT).

39. The method of any of claims 33-38, further comprising quantifying an amount of the compound in the subject.

40. The method of any of claims 33-39, wherein the method is used to obtain pharmacokinetic data of the compound.

41. The method of any of claims 33-40, wherein the method is used to obtain pharmacodynamic data of the compound.

42. The method of any of claims 33-41, wherein the method is used to monitor chemotherapy response in the subject.

43. The method of any of claims 33-42, wherein the subject has at least one mutation in BRCA1 or BRCA2.

44. The method of any of claims 33-43, wherein the cancer is breast cancer or ovarian cancer.

45. The method of any of claims 33-44, wherein the compound is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

46. The method of any of claims 33-46, wherein the administration is done prior to, concurrently with, or subsequent to an immunotherapeutic treatment.

47. The method of claim 46, wherein the immunotherapeutic treatment is selected from the group consisting of an immune checkpoint inhibitor, T-cell transfer therapy, an immune system modulator, a monoclonal antibody, and a treatment vaccine.
